(19) United States
(12) Patent Application Publication
(10) Pub. No.: US 2009/0317388 A1
(43) Pub. Date: Dec. 24, 2009

(54) Tl1a in treatment of disease

(76) Inventors: Linda Burkly, West Newton, MA (US); Anna Broodovsky, Cambridge, MA (US); Timothy Zheng, Boston, MA (US); Xingwen Dong, Wayne, PA (US)

Correspondence Address: MILLER, CANFIELD, PADDOCK AND STONE, PLC, 277 SOUTH ROSE STREET, SUITE 5000, KALAMAZOO, MI, 49007, US

(21) Appl. No.: 11/921,048

(22) PCT Filed: May 25, 2006
(86) PCT No.: PCT/US06/20234

§ 371 Date: Sep. 9, 2009

Related U.S. Application Data
(60) Provisional application No. 60/684,425, filed on May 25, 2005.

(51) Int. Cl. A61K 39/395; A61K 31/7105; A61K 38/16

(52) U.S. Cl. 424/134.1; 424/158.1; 514/44 A; 424/135.1; 424/133.1; 514/12

(57) ABSTRACT

Methods of modulating TL1A for the treatment of disease are disclosed.

### BACKGROUND

TL1A is a TNF superfamily member expressed by antigen presenting and endothelial cells. DR3, the receptor for TL1A, is expressed on activated lymphocytes and peripheral blood monocytes. (See Migone et al. (2002) Immunity 16:479-492). It has been reported that TL1A and DR3 expression are increased in the lamina propria of inflammatory bowel disease (IBD) intestinal tissue from both Crohn's disease (CD) and ulcerative colitis (UC) subjects, but a therapeutic effect of TL1A reduction has not been established. TL1A is localized in macrophages and in a small subset of CCR9+ T cells in CD specimens and in plasma cells in UC specimens; DR3 is primarily expressed on lymphocytes. TL1A costimulates secretion of the Th1 cytokine IFNgamma but not Th2 cytokines IL-4 and IL-10 by lamina propria lymphocytes (LPL) and synergizes with IL-12 and IL-18 for IFNgamma production in vitro. These data suggest that TL1A may play a role in the pathogenesis of Th1 mediated CD (Bamias et al., 2003, J Immunol. 171(9):4868-74; Prehn et al., 2004, Clin Immunol. 112(1):66-77).

### SUMMARY OF THE INVENTION

In one aspect, the invention features a method of treating multiple sclerosis (MS). The method includes administering, to a subject who has multiple sclerosis, an agent that blocks TL1A signaling, e.g., an agent that blocks TL1A interaction with DR3. The agent can be, e.g., a blocking anti-TL1A antibody or anti-DR3 antibody, a decoy DR3 polypeptide (e.g., a soluble DR3-Fc fusion protein), or a nucleic acid antagonist of TL1A or DR3.

In one embodiment, the agent is an antibody that is a full length IgG. In other embodiments, the agent is an antigen-binding fragment of a full length IgG, e.g., the agent is a single chain antibody, Fab fragment, F(ab′)2 fragment, Fd fragment, Fv fragment, or dAb fragment. In preferred embodiments, the antibody is a human, humanized or humaneered antibody or antigen-binding fragment thereof.

In one embodiment, the agent is a soluble form of a TL1A receptor (e.g., DR3). In some cases, the soluble form of the receptor is fused with a heterologous polypeptide, e.g., an antibody Fc region.

In one embodiment, the agent is administered in an amount sufficient to do one or more of the following: a) decrease severity or decrease frequency of relapse; b) prevent an increase in EDSS score, e.g., over a period of time, e.g., over 3 months, 6 months, a year or longer; c) decrease EDSS score (e.g., a decrease of greater than 1, 1.5, 2, 2.5, or 3 points, e.g., over at least three months, six months, one year, or longer); d) decrease the number of new MRI lesions; e)reduce the rate of appearance of new MRI lesions; and f) prevent an increase in MRI lesion area. The subject may be evaluated, before or after the administration, by MRI and/or neurological exam.

In one embodiment, the subject has relapsing-remitting (RR) MS, primary-progressive (PP) MS, secondary-progressive (SP) MS, or progressive-relapsing (PR) MS.

In one embodiment, the agent is administered in combination with another therapy for MS, e.g., copaxone; interferons, e.g., human interferon beta-1a (e.g., AVONEX® or Rebif®) and interferon beta-1b (BETASERON™; human interferon beta substituted at position 17); glatiramer acetate (also termed Copolymer 1, Cop-1; COPAXONE™); Tysabri® (natalizumab) ro another anti-VLA4 antibody, e.g., one that competes with or binds an epitope overlapping that of rituximab; Rituxan® (rituximab) or another anti-CD20 antibody, e.g., one that competes with or binds an overlapping epitope with rituximab; mixtoxantrone (NOVANTRONE®, Lederle); a corticosteroid.

In one embodiment, the agent is administered at a dose between 0.1-100 mg/kg, between 0.1-10 mg/kg, between 1 mg/kg -100 mg/kg, between 0.5-20 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

In another aspect, the invention features a method of treating ulcerative colitis (UC). The method includes administering, to a subject who has UC, an agent that blocks TL1A signaling, e.g., an agent that blocks TL1A interaction with DR3. The agent can be, e.g., a blocking anti-TL1A antibody or anti-DR3 antibody, a soluble decoy DR3 polypeptide (e.g., a soluble DR3-Fc fusion protein), or a nucleic acid antagonist of TL1A or DR3, such as an aptamer or antisense molecule.

In one embodiment, the agent is an anti-TL1A or anti-DR3 antibody that is a full length IgG. In other embodiments, the agent is an antigen-binding fragment of a full length IgG, e.g., the agent is a single chain antibody, Fab fragment, F(ab′)2 fragment, Fd fragment, Fv fragment, or dAb fragment. In preferred embodiments, the antibody is a human, humanized or humaneered antibody or antigen-binding fragment thereof.

In one embodiment, the agent is a soluble form of a TL1A receptor (e.g., DR3). In some cases, the soluble form of the receptor is fused with a heterologous polypeptide, e.g., an antibody Fc region.

In one embodiment, the agent is administered in an amount sufficient to do one or more of the following: a) decrease severity or decrease frequency of colitis flare-ups; b) prevent or decrease the extent of weight loss; (c) improve the presence or extent of ulcers or inflammation, e.g., over a period of time, e.g., over 3 months, 6 months, a year or longer. The subject may be evaluated, before or after the administration, with one or more of the following: colonoscopy with or without biopsy, barium enema, CBC blood test, sedimentation rate (ESR), CRP (C-reactive protein) test.

In one embodiment, the subject has an acute flare-up of UC.

In one embodiment, the agent is administered in combination with another therapy for UC, e.g., corticosteroids to reduce inflammation; aminosalicylates; immunosuppressants, such as azathioprine; 6-MP, cyclosporine, and methotrexate.

In one embodiment, the agent is administered at a dose between 0.1-100 mg/kg, between 0.1-10 mg/kg, between 1 mg/kg-100 mg/kg, between 0.5-20 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

In another aspect, the invention features methods for modulating an innate immunity response in a subject by modulating TL1A signaling.

In one aspect, a method is provided to reduce an innate immunity response in a subject in need thereof. The method includes administering, to a subject who has a hyper-responsive innate immunity response, an agent that blocks TL1A signaling, e.g., an agent that blocks TL1A interaction with DR3. The agent can be, e.g., a blocking anti-TL1A antibody, anti-DR3 antibody, or a soluble DR3 (e.g., a soluble DR3-Fc fusion protein).

In some embodiments, the subject in need of reducing an innate immunity response has an autoimmune disease, e.g., rheumatoid arthritis, SLE, Grave's Disease, Wegener's granulomatosis, Sjogren's syndrome, scleroderma, type 1 diabetes mellitus; a neuroinflammatory disease, e.g., MS, ALS, Alzheimer's Disease.

In one embodiment, the agent is administered in an amount sufficient to reduce the number and/or activity of innate immune cell types such as macrophages, monocytes, dendritic cells and neutrophils. In one embodiment, the agent is administered in an amount sufficient to reduce production by such innate immune cell types of proinflammatory cytokines, e.g., IL-6, IL-12, IL-23, TNF, IFNgamma, IL-1, IL-8, IL-10, type 1 interferons, IL-11, IL-23, Il-27, GM-CSF, G-CSF, M-CSF and chemokines including but not limited to MIP-1alpha, MIP-1beta, CXCL11, RANTES, TARC, MCP-5, eotaxin and those referenced herein (e.g., Rot and von Adrian, 2004, Ann. Rev. Immunol. 22:891-928; Moser et al., 2004, Trends in Immunol 25: 75-84).

In one embodiment, the method also includes evaluating the subject for a marker of innate immunity response, e.g., evaluating the subject for numbers and/or activity (e.g., phagocytic activity) of immune cells (i.e. white blood cells, lymphocytes, neutrophils, monocytes), or macrophage release of proinflammatory cytokines, e.g., as described hereinabove. The evaluation can be performed before and/or after the administration. In one embodiment, the subject is evaluated for such a marker periodically (e.g., at least 2 times) over a period of time after the administration.

In one embodiment, the agent is an antibody that is a full length IgG. In other embodiments, the agent is an antigen-binding fragment of a full length IgG, e.g., the agent is a single chain antibody, Fab fragment, F(ab′)2 fragment, Fd fragment, Fv fragment, or dAb fragment. In preferred embodiments, the antibody is a human, humanized or humaneered antibody or antigen-binding fragment thereof. The antibody can be, e.g., an anti-TL1A antibody or an anti-DR3 antibody.

In one embodiment, the agent is a soluble form of a TL1A receptor (e.g., DR3), e.g., a polypeptide. In some cases, the soluble form of the receptor is fused with a heterologous polypeptide, e.g., an antibody Fc region.

In one embodiment, the agent is administered at a dose between 0.01-100 mg/kg, between 0.01-10 mg/kg, between 0.01 mg/kg-1 mg/kg, between 0.05-10 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

Conditions which may benefit from reducing the innate immunity response include conditions in which innate immunity is hyper-responsive, e.g., conditions in which innate immune response to a pathogen leads to an inflammatory disorder, e.g., to an acute flare-up of an inflammatory disorder. In one embodiment, the subject has an inflammatory disease or autoimmune disease and is at risk for acute flare-ups, e.g., an acute flare-up of IBD or colitis.

In another aspect, a method is provided to enhance an innate immunity response in a subject in need thereof. The method includes administering an agent that enhances TL1A signaling in an amount that stimulates innate immunity, e.g., an amount that causes an enhancement in resistance to, reduction in susceptibility to, or decrease in pathogenic effects of, an infective agent such as a bacterial or viral infection; or a cancer cell. An agent that enhances TL1A signaling can be, e.g., a soluble TL1A, a multimerized TL1A such as a trimerized TL1A (e.g., as described for CD40L in Morris et al. (1999) J. Biol. Chem. 274:418-423), and an anti-DR3 agonist antibody.

In one embodiment, the agent is administered in an amount sufficient to increase the number and/or activity of innate immune cell types such as macrophages, monocytes, dendritic cells and neutrophils. In one embodiment, the agent is administered in an amount sufficient to increase production by such innate immune cell types of proinflammatory cytokines, e.g., IL-6, IL-1 2, IL-23, TNF, IFNgamma, IL-1, IL-8, IL-10, type 1 interferons, IL-11, IL-23, Il-27, GM-CSF, G-CSF, M-CSF and chemokines including but not limited to MIP-1alpha, MIP-1beta, CXCL11, RANTES, TARC, MCP-5, eotaxin and those referenced herein (e.g., Rot and von Adrian, 2004, Ann. Rev. Immunol. 22:891-928; Moser et al., 2004, Trends in Immunol 25: 75-84)

In one embodiment, the method also includes evaluating the subject for a marker of innate immunity response, e.g., evaluating the subject for numbers and/or activity (e.g., phagocytic activity) of immune cells (i.e. white blood cells, lymphocytes, neutrophils, monocytes), or macrophage release of proinflammatory cytokines, e.g., as described hereinabove. The evaluation can be performed before and/or after the administration. In one embodiment, the subject is evaluated for such a marker periodically (e.g., at least 2 times) over a period of time after the administration.

In one embodiment, the agent is an anti-DR3 agonist antibody that is a full length IgG. In other embodiments, the agent is an antigen-binding fragment of a full length IgG, e.g., the agent is a single chain antibody, Fab fragment, F(ab′)2 fragment, Fd fragment, Fv fragment, or dAb fragment. In preferred embodiments, the antibody is a human, humanized or humaneered antibody or antigen-binding fragment thereof. The antibody can be, e.g., an anti-DR3 antibody.

In one embodiment, the agent is a soluble form of TL1A. In some cases, the soluble TL1A is fused with a heterologous polypeptide, e.g., an antibody Fc region.

In one embodiment, the agent is administered at a dose between 0.1-100 mg/kg, between 0.1-10 mg/kg, between 1 mg/kg-100 mg/kg, between 0.5-20 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

Conditions which may benefit from enhanced innate immunity response include conditions associated with inadequate innate immunity response including hypo-responsiveness to LPS, susceptibility to infection or sepsis (e.g., by gram-negative bacteria), susceptibility to chronic airway disease, susceptibility to asthma, susceptibility to arthritis, susceptibility to pyelonephritis, susceptibility to gall bladder disease, susceptibility to pneumonia, susceptibility to bronchitis, susceptibility to chronic obstructive pulmonary disease, severity of cystic fibrosis, and susceptibility to local and systemic inflammatory conditions, e.g., systemic inflammatory response syndrome (SIRS), local gram negative bacterial infection, or acute respiratory distress syndrome (ARDS), and susceptibility to cancer or decreased ability of the innate immune system to reject cancer cells. In some embodiments, certain patients can benefit from enhanced innate immunity response, e.g., (i) patients having opportunistic infections, pneumocystis infection, cytomegalovirus infection, herpes virus infection, mycobacterium infection, or human immunodeficiency virus (HIV) infection; (ii) patients exposed to radiation or one or more chemotherapeutic antiproliferative drugs; (iii) patients who have cancer; (iv) patients having chronic respiratory disease or upper airways disease, (e.g., sinusitis or parasinusitis, rhinovirus or influenza infection, pleuritis, and the like); (v) patients having chronic eye-ear-nose or throat infections (e.g., otitis media, conjunctivitis, uveitis or keratitis); (vi) patients having bronchial allergy and/or asthma; (vii) patients having a chronic liver infection (e.g., chronic hepatitis); and (viii) other immunocompromised patients.

In one embodiment, the subject has cancer or has susceptibility to cancer. For example, the subject has a family history of cancer or carries a genetic marker for susceptibility to cancer, such as BRCA1 or BRCA2, or one or more other genes that are causally implicated in oncogenesis. A census of such genes is provided in Futreal et al. (2004) Nature Reviews Cancer 4:177-183.

In one embodiment, the subject has defective phagocytic function, e.g., defective macrophage function. In another embodiment, the subject has chronic granulomatous disease. In one embodiment, the subject has defective phagocytic function and has Alzheimer's Disease.

As used herein, the term “treating” refers to administering a therapy in an amount, manner, and/or mode effective to improve or prevent a condition, symptom, or parameter associated with a disorder or to prevent onset, progression, or exacerbation of the disorder (including secondary damage caused by the disorder), to either a statistically significant degree or to a degree detectable to one skilled in the art. Accordingly, treating can achieve therapeutic and/or prophylactic benefits. An effective amount, manner, or mode can vary depending on the subject and may be tailored to the subject.

Each of the limitations of the invention can encompass various embodiments of the invention. It is, therefore anticipated that each of the limitations of the invention involving any one element or combinations of elements can be included in each aspect of the invention.

### BRIEF DESCRIPTION OF THE FIGURES

FIG. 1 is a restriction map of the murine Tnfsf15 locus and the thymidine kinase (TK) and neomycin (neo) containing targeting construct derived from it. Restriction enzyme sites indicated are: E-EcoRI, X-XbaI, Bg-BglII, Ba-BamHI. Exons are represented as black boxes, arrows indicate direction of transcription. B. RT-PCR analysis of TL1A mRNA in TL1A−/− and WT kidneys.

FIG. 2 shows EAE clinical course in TL1A−/− animals. A. EAE disease course in C57BL/6 TL1A^{-/-} (round symbols) and wild type (square symbols) mice. Mice were immunized with MOG₃₅₋₅₅ and pertussis toxin as described in Examples. Values represent the mean clinical score for each group, error bars are SEM. Disease course representative of 4 independent experiments, n=7-10 animals per group. B. EAE statistical parameters for results shown in A. Day of onset was calculated for diseased animals only. p-values are shown for TL1A−/− vs. WT group based on the Mann-Whitney non-parametric test.

FIG. 3 shows the MOG-specific cytokine response. A-E. Cytokine secretion in wild type (white bars) and TL1A^{-/-} (grey bars) lymph node cultures. Cells were cultured as in FIG. 5B, except 50 ug/ml MOG peptide was used. 72 hr supernatants from 5 individuals/group were analyzed: A-IFNγ, B-GM-CSF, C-TNFα, D-IL-4, E-IL-5. Results shown are mean values ± SEM. Asterisks indicate statistically significant differences (two-tail t-test p<0.05).

FIG. 4 shows that deficiency of TL1A protects against the development of DSS-induced colitis. 8-12 week old female C57BL/6 TL1A−/− (round symbols) and WT animals (square symbols) were fed with 3.5% (wt/vol) DSS dissolved in water for 5 days (days 0-4). DSS is stopped and normal drinking water restored during days 5-13. Body weight, stool consistency, and the presence of occult or visible blood in the stool were determined daily. Disease Score (A) is the combined scores of weight loss, stool consistency and bleeding divided by 3. Values represent the mean clinical score for each group, error bars are SEM. Disease course representative of 3 independent experiments, n=10-11 animals per group. Statistical analysis was performed using Mann-Whitney non-parametric test. *, p<0.05 for comparison of TL1A^{-/-} with WT mice.

FIG. 5 shows that TL1A deficient mice develop fewer colonic ulcers, less epithelial damage and less cell infiltration during DSS treatment. 8-12 week old female C57BL/6 TL1A^{-/-} (open bars) and WT animals (closed bars) were fed with 3.5% (wt/vol) DSS dissolved in water for 5 days (days 0-4). DSS is stopped and normal drinking water restored during days 5-13. (n=10-13 animals per group). Mice were sacrificed at day 5, 11 and 13. Colons were paraffin embedded and stained with H&E. The extent of mucosal ulceration (A), epithelial damage (C), inflammatory cell infiltration into the colonic tissue (D), and total histological score (the combined scores of epithelium cell damage and cell infiltration) (B) was quantified as described in Materials and Methods. Statistical analysis was performed using Mann-Whitney non-parametric U test. *, p<0.05 for comparison of TL1A^{-/-} with WT mice, and p=0.09 for ulcer index on day 11 not including results from two WT mice that did not survive the DSS treatment. Statistical analysis for C and D are not shown.

### DETAILED DESCRIPTION

The inventors have discovered that antagonizing (e.g. blocking) the TL1A pathway is effective to reduce pathogenesis in animal models of multiple sclerosis and ulcerative colitis. The data also supports a role for TL1A in the innate immunity response, e.g., in the pathogenesis of ulcerative colitis.

TL1A (TNFSF15) is the ligand for DR3 (TNFRSF12) and is a member of the tumor necrosis factor superfamily (TNFSF). The amino acid sequence of human TL1A is shown below.

(SEQ ID NO: 1)

| | |
|---|---|
| 1 | MAEDLGLSFG ETASVEMLPE HGSCRPKARS SSARWALTCC |
| | LVLLPFLAGL TTYLLVSQLR |
| 61 | AQGEACVQFQ ALKGQEFAPS HQQVYAPLRA DGDKPRAHLT |
| | VVRQTPTQHF KNQFPALHWE |
| 121 | HELGLAFTKN RMNYTNKFLL IPESGDYFIY SQVTFRGMTS |
| | ECSEIRQAGR PNKPDSITVV |
| 181 | ITKVTDSYPE PTQLLMGTKS VCEVGSNWFQ PIYLGAMFSL |
| | QEGDKLMVNV SDISLVDYTK |
| 241 | EDKTFFGAFL L |

A soluble TL1A lacks the transmembrane domain and cytosolic domain. It can include amino acids 93 to 251 of SEQ ID NO:1, or an N- or C-terminal truncation thereof (e.g., a truncation lacking up to 10 (e.g., up to 8, 6, 4, 2) residues at the N- and/or C-terminal end of amino acids 93-251 of SEQ ID NO: 1), and having DR3 binding activity. In one embodiment, a soluble TL1A includes amino acids 73-251 of SEQ ID NO: 1; amino acids 103-251 of SEQ ID NO:1, amino acids 93-251 of SEQ ID NO:1; amino acids 93-245 of SEQ ID NO: 1. Also included are polypeptides that include a sequence that has at least 95% identity (e.g., 96%, 97%, 98%, 99% identity) to soluble TL1A, e.g., to amino acids 103-251 of SEQ ID NO: 1, and has DR3 binding activity.

The amino acid sequence of DR3 (the receptor for TL1A) is shown below (see Bodmer et al. (1997) Immunity 6:79-88).

(SEQ ID NO: 2)

| | |
|---|---|
| 1 | MEQRPRGCAA VAAALLLVLL GARAQGGTRS PRCDCAGDFH |
| | KKIGLFCCRG CPAGHYLKAP |
| 61 | CTEPCGNSTC LVCPQDTFLA WENHHNSECA RCQACDEQAS |
| | QVALENCSAV ADTRCGCKPG |
| 121 | WFVECQVSQC VSSSPFYCQP CLDCGALHRH TRLLCSRRDT |
| | DCGTCLPGFY EHGDGCVSCP |
| 181 | TSTLGSCPER CAAVCGWRQM FWVQVLLAGL VVPLLLGATL |
| | TYTYRHCWPH KPLVTADEAG |
| 241 | MEALTPPPAT HLSPLDSAHT LLAPPDSSEK ICTVQLVGNS |
| | WTPGYPETQE ALCPQVTWSW |
| 301 | DQLPSRALGP AAAPTLSPES PAGSPAMMLQ PGPQLYDVMD |
| | AVPARRWKEF VRTLGLREAE |
| 361 | IEAVEVEIGR FRDQQYEMLK RWRQQQPAGL GAVYAALERM |
| | GLDGCVEDLR SRLQRGP |

Residues 1-24 of SEQ ID NO:2 correspond to the signal peptide of DR3; residues 25-206 of SEQ ID NO:2 correspond to the extracellular domain of the mature protein; residues 207-226 of SEQ ID NO:2 correspond to the transmembrane domain. The cytoplasmic domain includes a death domain (DD) at residues 335-419 of SEQ ID NO:2. A soluble decoy DR3 lacks the transmembrane domain and cytosolic domain, e.g., it includes residues 25-181 of SEQ ID NO:2 (the extracellular domain), or a functional N- or C-terminal truncation thereof, e.g., a truncation lacking 10 (e.g., 9, 8, 7, 6, 5, 4, 3, 2) or fewer residues at the N- and/or C-terminus. Examples of soluble decoy DR3 polypeptides include polypeptides including amino acids 25-181 of SEQ ID NO:2, amino acids 25-191 of SEQ ID NO:2, amino acids 40-206 of SEQ ID NO:2, amino acids 30-200 of SEQ ID NO:2, amino acids 40-181 of SEQ ID NO:2. Also included are polypeptides having at least 95% identity (e.g., 96%, 97%, 98%, 99% identity) to a functional portion of the extracellular domain of DR3 (residues 25-181 of SEQ ID NO:2), and having TL1A binding activity.

### Antibodies

Antibodies that block TL1A function, e.g., antibodies that bind to TL1A or DR3 can be generated by immunization, e.g., using an animal, or by in vitro methods such as phage display. All or part of TL1A or DR3 can be used as an immunogen. For example, the extracellular region of TL1A or DR3 can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XenoMouse™, Green et al. Nature Genetics 7:13-21 (1994), US 2003-0070185, U.S. Pat. No. 5,789,650, and WO 96/34096.

Non-human antibodies to TL1A or DR3 can also be produced, e.g., in a rodent. The non-human antibody can be humanized, e.g., as described in U.S. Pat. No. 6,602,503, EP 239 400, U.S. Pat. No. 5,693,761, and U.S. Pat. No. 6,407,213.

EP 239 400 (Winter et al.) describes altering antibodies by substitution (within a given variable region) of their complementarity determining regions (CDRs) for one species with those from another. CDR-substituted antibodies can be less likely to elicit an immune response in humans compared to true chimeric antibodies because the CDR-substituted antibodies contain considerably less non-human components. (Riechmann et al., 1988, Nature 332, 323-327; Verhoeyen et al., 1988, Science 239, 1534-1536). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Queen et al. (Proc. Natl. Acad. Sci. U.S.A. 86:10029-33, 1989) and WO 90/07861 have described a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and 5,530,101. Tempest et al., 1991, Biotechnology 9:266-271, utilize, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from x-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (preferably at least five, ten, twelve, or all) of the following positions: (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies can be produced, e.g., using in vitro-primed human splenocytes, as described by Boemer et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; US 2003-0232333).

### Antibody Production

Antibodies can be produced in prokaryotic and eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies, particularly full length antibodies, e.g., IgG's, are produced in mammalian cells. Exemplary mammalian host cells for recombinant expression include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). Exemplary selectable marker genes include the dihydrofolate reductase (DHFR) gene (for use in dhfr-host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2 domain. This asparagine is the site for modification with biantennary-type oligosaccharides. This glycosylation participates in effector functions mediated by Fc □ receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the antibody of interest, e.g., an antibody described herein. The antibody can be purified from the milk, or for some applications, used directly.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, an antibody can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, an antibody can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

### Soluble Receptors

Some embodiments of the invention involve the use of a soluble TL1A receptor, e.g., a soluble DR3 receptor or fusion protein. For example, a protein including a TL1A-binding portion of the extracellular domain of DR3 can be fused to an Fc region, i.e., to the C-terminal portion of an Ig heavy chain constant region. Such a fusion may have improved solubility and/or in vivo stability relative to a soluble DR3 alone. The Fc region used can be an IgA, IgD, or IgG Fc (e.g., an IgG1 or IgG4 Fc) region (hinge-CH2-CH3). Alternatively, it can be an IgE or IgM Fc region (hinge-CH2-CH3-CH4). Materials and methods for constructing and expressing DNA encoding Fc fusions are known in the art.

The DR3 portion of the fusion protein preferably includes at least a portion of the extracellular region of DR3 (a TL1A binding portion) and preferably lacks a transmembrane domain, such that the DR3 moiety is soluble. The soluble DR3 is typically comprised of amino acids 1-199 or a functional (e.g., TL1A binding) fragment thereof of SEQ ID NO:2.

The signal sequence is a polynucleotide that encodes an amino acid sequence that initiates transport of a protein across the membrane of the endoplasmic reticulum. Signal sequences useful for constructing a fusion protein include antibody light chain signal sequences, e.g., antibody 14.18 (Gillies et. al., 1989, J. Immunol. Meth., 125:191-202), antibody heavy chain signal sequences, e.g., the MOPC141 antibody heavy chain signal sequence (Sakano et al., 1980, Nature 286:5774). Alternatively, other signal sequences can be used. See, for example, Watson, 1984, Nucleic Acids Research 12:5145). The signal peptide is usually cleaved in the lumen of the endoplasmic reticulum by signal peptidases. This results in the secretion of a fusion protein containing the Fc region and the TL1A or DR3 moiety.

In some embodiments the DNA sequence encodes a proteolytic cleavage site between the secretion cassette and the DR3 moiety. A cleavage site provides for the proteolytic cleavage of the encoded fusion protein, thus separating the Fc domain from the target protein. Useful proteolytic cleavage sites include amino acids sequences recognized by proteolytic enzymes such as trypsin, plasmin, thrombin, factor Xa, or enterokinase K. The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., 1991, Biochim. Biophys. Acta 1088:712; and Lo et al., 1998, Protein Engineering 11:495-500. An appropriate host cell can be transformed or transfected with a DNA that encodes a TL1A or DR3 polypeptide, and is used for the expression and secretion of the TL1A or DR3 polypeptide. Preferred host cells include immortal hybridoma cells, myeloma cells, 293 cells, Chinese hamster ovary (CHO) cells, Hela cells, and COS cells.

Certain sites preferably can be deleted from the Fc region during the construction of the secretion cassette. For example, since coexpression with the light chain is unnecessary, the binding site for the heavy chain binding protein, Bip (Hendershot et al., 1987, Immunol. Today 8:111-114), can be deleted from the CH2 domain of the Fc region of IgE, such that this site does not interfere with the efficient secretion of the immunofusin. Transmembrane domain sequences, such as those present in IgM, can be deleted.

The IgG1Fc region is one example. Alternatively, the Fc region of the other subclasses of immunoglobulin gamma (gamma-2, gamma-3 and gamma-4) can be used in the secretion cassette. The IgG1 Fc region of immunoglobulin gamma-1 is preferably used in the secretion cassette includes the hinge region (at least part), the CH2 region, and all or part of the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., 1990, Hum. Antibod. Hybridomas, 1:47. In some embodiments, the Fc regions of IgA, IgD, IgE, or IgM, are used.

DR3 fusion proteins can be constructed in several different configurations. In one configuration the C-terminus of the DR3 moiety is fused directly to the N-terminus of the Fc moiety. In a slightly different configuration, a short polypeptide, e.g., 2-10 amino acids, is incorporated into the fusion between the N-terminus of the DR3 moiety and the C-terminus of the Fc moiety. Such a linker can provide conformational flexibility, which may improve biological activity in some circumstances. If a sufficient portion of the hinge region is retained in the Fc moiety, the DR3-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

### Polynucleotide Antagonists

Some methods described herein relate to administering an effective amount of a TL1A or DR3 polynucleotide antagonist. The polynucleotide antagonist prevents expression of the target gene (knockdown). Such polynucleotide antagonists include, but are not limited to antisense molecules, ribozymes, aptamers, siRNA, shRNA and RNAi. Typically, such binding molecules are separately administered to the subject (see, for example, O'Connor (1991) Neurochem. 56:560), but such binding molecules may also be expressed in vivo from polynucleotides taken up by a host cell and expressed in vivo. See also Oligodeoxynucleotides as Antisense Inhibitors of Gene Expression, CRC Press, Boca Raton, Fla. (1988).

RNAi

RNAi refers to the expression of an RNA which interferes with the expression of the targeted mRNA. Specifically, the RNAi silences a targeted gene via interacting with the specific mRNA (e.g. TL1A or DR3) through a siRNA (short interfering RNA). The ds RNA complex is then targeted for degradation by the cell. Additional RNAi molecules include Short hairpin RNA (shRNA); also short interfering hairpin. The shRNA molecule contains sense and antisense sequences from a target gene connected by a loop. The shRNA is transported from the nucleus into the cytoplasm, it is degraded along with the mRNA. Pol III or U6 promoters can be used to express RNAs for RNAI.

RNAi is mediated by double stranded RNA (dsRNA) molecules that have sequence-specific homology to their “target” mRNAs (Caplen et al. (2001) Proc Natl Acad Sci USA 98:9742-9747). Biochemical studies in Drosophila cell-free lysates indicates that the mediators of RNA-dependent gene silencing are 21-25 nucleotide “small interfering” RNA duplexes (siRNAs). Accordingly, siRNA molecules are advantageously used in methods described herein. The siRNAs are derived from the processing of dsRNA by an RNase known as DICER (Bernstein et al. (2001) Nature 409:363-366). It appears that siRNA duplex products are recruited into a multi-protein siRNA complex termed RISC (RNA Induced Silencing Complex). Without wishing to be bound by any particular theory, it is believed that a RISC is guided to a target mRNA, where the siRNA duplex interacts sequence-specifically to mediate cleavage in a catalytic fashion (Bernstein et al. (2001) Nature 409:363-366; Boutla et al. (2001) Curr Biol 11: 1776-1780).

RNAi is contemplated as a therapeutic modality, such as inhibiting or blocking the infection, replication and/or growth of viruses (Gitlin et al. (2002) Nature 418:379-380; Capodici et al. (2002) J Immunol 169:5196-5201), and reducing expression of oncogenes (Scherr et al (2003) Blood 101(4):1566-9). RNAi has been used to modulate gene expression in mammalian (mouse) and amphibian (Xenopus) embryos (Calegari et al., Proc Natl Acad Sci USA 99:14236-14240, 2002; and Zhou, et al., Nucleic Acids Res 30:1664-1669, 2002), and in postnatal mice (Lewis et al., Nat Genet 32:107-108, 2002), and to reduce trangsene expression in adult transgenic mice (McCaffrey et al., Nature 418:38-39, 2002). Methods have been described for determining the efficacy and specificity of siRNAs in cell culture and in vivo (see, e.g., Bertrand et al., Biochem Biophys Res Commun 296:1000-1004, 2002; Lassus et al., Sci STKE 2002(147):PL13, 2002; and Leirdal et al., Biochem Biophys Res Commun 295:744-748, 2002).

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

References regarding siRNA include: Bernstein et al., Nature 409:363-366, 2001; Boutla et al., Curr Biol 11:1776-1780, 2001; Cullen, Nat Immunol. 3:597-599, 2002; Caplen et al., Proc Natl Acad Sci USA 98:9742-9747, 2001; Hamilton et al., Science 286:950-952, 1999; Nagase et al., DNA Res. 6:63-70, 1999; Napoli et al., Plant Cell 2:279-289, 1990; Nicholson et al., Mamm. Genome 13:67-73, 2002; Parrish et al., Mol Cell 6:1077-1087, 2000; Romano et al., Mol Microbiol 6:3343-3353, 1992; Tabara et al., Cell 99:123-132, 1999; and Tuschl, Chembiochem. 2:239-245, 2001.

Paddison et al. (Genes & Dev. 16:948-958, 2002) have used small RNA molecules folded into hairpins as a means to effect RNAi. Accordingly, such short hairpin RNA (shRNA) molecules are also advantageously used in the methods of the invention. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the dsRNA products of the DICER RNase and, in any event, have the same capacity for inhibiting expression of a specific gene. In some embodiments of the invention, the shRNA is expressed from a lentiviral vector, e.g., pLL3.7.

Antisense

Antisense technology can be used to control gene expression through antisense DNA or RNA, or through triple-helix formation. Antisense techniques are discussed for example, in Okano, J. Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ non-coding portion of a polynucleotide that encodes TL1A or DR3 may be used to design an antisense RNA oligonucleotide of from about 10 to 40 base pairs in length. A DNA oligonucleotide is designed to be complementary to a region of the gene involved in transcription thereby preventing transcription and the production of the target protein. The antisense RNA oligonucleotide hybridizes to the mRNA in vivo and blocks translation of the mRNA molecule into the target polypeptide.

In one embodiment, antisense nucleic acids specific for the TL1A or DR3 gene are produced intracellularly by transcription from an exogenous sequence. For example, a vector or a portion thereof, is transcribed, producing an antisense nucleic acid (RNA). Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the antisense molecule, can be by any promoter known in the art to act in vertebrate, preferably human cells, such as those described elsewhere herein. Absolute complementarity of an antisense molecule, although preferred, is not required. A sequence complementary to at least a portion of an RNA encoding TL1A or DR3, means a sequence having sufficient complementarity to be able to hybridize with the RNA, forming a stable duplex; or triplex formation may be assayed. The ability to hybridize will depend on both the degree of complementarity and the length of the antisense nucleic acid. Generally, the larger the hybridizing nucleic acid, the more base mismatches it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of a messenger RNA, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature 372:333-335 (1994). Thus, oligonucleotides complementary to either the 5′- or 3′-non-translated, non-coding regions could be used in an antisense approach to inhibit translation of TL1A or DR3. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Polynucleotides for use the therapeutic methods disclosed herein can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. USA. 86:6553-6556 (1989); Lemaitre et al., Proc. Natl. Acad. Sci. USA 84:648-652 (1987)); PCT Publication No. WO88/098 10, published Dec. 15, 1988) or the blood-brain barrier (see, e.g., PCT Publication No. WO89/10134, published Apr. 25, 1988), hybridization-triggered cleavage agents. (See, e.g., Krol et al., BioTechniques 6:958-976 (1988)) or intercalating agents. (See, e.g., Zon, Pharm. Res. 5:539-549(1988)). To this end, the oligonucleotide may be conjugated to another molecule, e.g., a peptide, hybridization triggered cross-linking agent, transport agent, hybridization-triggered cleavage agent, etc.

An antisense oligonucleotide for use in the therapeutic methods disclosed herein may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta-D-mannosylqueosine, 5′methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3(3-amino-3-N-2-carboxypropyl)uracil, (acp3)w, and 2,6-diaminopurine.

An antisense oligonucleotide for use in the therapeutic methods disclosed herein may also comprise at least one modified sugar moiety selected from the group including, but not limited to, arabinose, 2-fluoroarabinose, xylulose, and hexose.

In yet another embodiment, an antisense oligonucleotide for use in the therapeutic methods disclosed herein comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof. In yet another embodiment, an antisense oligonucleotide for use in the therapeutic methods disclosed herein is an alpha-anomeric oligonucleotide. An alpha-anomeric oligonucleotide forms specific double-stranded hybrids with complementary RNA in which, contrary to the usual situation, the strands run parallel to each other (Gautier et al., Nucl. Acids Res. 15:6625-6641(1987)). The oligonucleotide is a 2′-.beta.-methylribonucleotide (Inoue et al., Nucl. Acids Res. 15:6131-6148(1987)), or a chimeric RNA-DNA analogue (Inoue et al., FEBS Lett. 215:327-330(1987)).

Polynucleotides may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), methylphosphonate oligonucleotides can be prepared by use of controlled pore glass polymer supports (Sarin et al., Proc. Natl. Acad. Sci. USA. 85:7448-7451(1988)), etc. Polynucleotide compositions for use in the therapeutic methods disclosed herein further include catalytic RNA, or a ribozyme (See, e.g., PCT International Publication WO 90/11364, published Oct. 4, 1990; Sarver et al., Science 247:1222-1225 (1990). The use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). Preferably, the ribozyme is engineered so that the cleavage recognition site is located near the 5′ end of the target mRNA; i.e., to increase efficiency and minimize the intracellular accumulation of non-functional mRNA transcripts.

Ribozymes

As in the antisense approach, ribozymes for use in the therapeutic methods disclosed herein can be composed of modified oligonucleotides (e.g. for improved stability, targeting, etc.) and may be delivered to cells which express TL1A or DR3 in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous TL1A or DR3 messages and inhibit translation. Since ribozymes unlike antisense molecules, are catalytic, a lower intracellular concentration is required for efficiency.

Aptamers

Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (See Ulrich (2006) Handb Exp Pharmacol. 173:305-26). Macugen® is an approved aptamer therapeutic which is also the first anti-angiogenic agent approved for a common eye disorder.

### Pharmaceutical Compositions

An agent described herein can be formulated as a pharmaceutical composition. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Agents described herein can be formulated according to standard methods. Pharmaceutical formulation is a well-established art, and is further described in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

In one embodiment, an agent (e.g., an antibody) can be formulated with excipient materials, such as sodium chloride, sodium dibasic phosphate heptahydrate, sodium monobasic phosphate, and polysorbate 80. It can be provided, for example, in a buffered solution at a concentration of about 20 mg/ml and can be stored at 2-8° C. Pharmaceutical compositions may also be in a variety of other forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration.

The composition can be formulated as a solution, microemulsion, dispersion, liposome, or other ordered structure suitable to high drug concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

### Administration

An agent described herein (e.g., an antibody) can be administered to a subject, e.g., a human subject, by a variety of methods. For many applications, the route of administration is one of: intravenous injection or infusion, subcutaneous injection, or intramuscular injection. An antibody can be administered as a fixed dose, or in a mg/kg dose, but preferably as a fixed dose. The antibody can be administered intravenously (IV), subcutaneously (SC) or intramuscularly (IM).

Dosage regimens are adjusted to provide the desired response, e.g., a therapeutic response. For example, doses in the range of 0.1-100 mg/kg, 1 mg/kg-100 mg/kg, 0.5-20 mg/kg, 0.1-10 mg/kg or 1-10 mg/kg can be administered. A particular dose may be administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

In certain embodiments, the active agent may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Pharmaceutical compositions can be administered with medical devices. For example, pharmaceutical compositions can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, other such implants, delivery systems, and modules are also known.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

A pharmaceutical composition may include a “therapeutically effective amount” of an agent described herein. A therapeutically effective amount of an agent may also vary according to factors such as the disease state, age, sex, and weight of the individual, and the ability of the compound to elicit a desired response in the individual, e.g., amelioration of at least one disorder parameter, e.g., a multiple sclerosis parameter, or amelioration of at least one symptom of the disorder, e.g., multiple sclerosis. A therapeutically effective amount is also one in which any toxic or detrimental effects of the composition is outweighed by the therapeutically beneficial effects.

### Multiple Sclerosis

Multiple sclerosis (MS) is a central nervous system disease that is characterized by inflammation and loss of myelin sheaths. MS may be identified by criteria establishing a diagnosis of clinically definite MS as defined by the workshop on the diagnosis of MS (Poser et al., Ann. Neurol. 13:227, 1983). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al., 2001, Recommended diagnostic criteria for multiple sclerosis: guidelines from the International Panel on the Diagnosis of Multiple Sclerosis, Ann Neurol 50:121-127). The McDonald criteria include the use of MRI evidence of CNS impairment over time to be used in diagnosis of MS, in the absence of multiple clinical attacks. Effective treatment of multiple sclerosis may be evaluated in several different ways. The following parameters can be used to gauge effectiveness of treatment. Two exemplary criteria include: EDSS (extended disability status scale), and appearance of exacerbations on MRI (magnetic resonance imaging). The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step indicates an effective treatment (Kurtzke, Ann. Neurol. 36:573-79, 1994).

MRI can be used to measure active lesions using gadolinium-DTPA-enhanced imaging (McDonald et al. Ann. Neurol. 36:14, 1994) or the location and extent of lesions using T2-weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of MS lesions can be determined by radiologists. Areas of lesions can be outlined and summed slice by slice for total lesion area. Three analyses may be done: evidence of new lesions, rate of appearance of active lesions, percentage change in lesion area (Paty et al., Neurology 43:665, 1993). Improvement due to therapy can be established by a statistically significant improvement in an individual patient compared to baseline or in a treated group versus a placebo group.

Exemplary symptoms associated with multiple sclerosis, which may be improved with the methods described herein, include: optic neuritis, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, uhthoff's symptom, gastroesophageal reflux, and sleeping disorders.

Each case of MS displays one of several patterns of presentation and subsequent course. Most commonly, MS first manifests itself as a series of attacks followed by complete or partial remissions as symptoms mysteriously lessen, only to return later after a period of stability. This is called relapsing-remitting (RR) MS. Primary-progressive (PP) MS is characterized by a gradual clinical decline with no distinct remissions, although there may be temporary plateaus or minor relief from symptoms. Secondary-progressive (SP) MS begins with a relapsing-remitting course followed by a later primary-progressive course. Rarely, patients may have a progressive-relapsing (PR) course in which the disease takes a progressive path punctuated by acute attacks. PP, SP, and PR are sometimes lumped together and called chronic progressive MS.

### Innate Immunity

Innate immunity is the body's first, generalized line of defense against pathogens, which includes the rapid inflammation of tissues that takes place shortly after injury or infection, hindering the entrance and spread of disease. Innate immune responses are effected by a wide array of effector cells, including phagocytic cells (neutrophils, monocytes, macrophages and dendritic cells), cells that release inflammatory mediators (basophils, mast cells, and eosinophils), and natural killer cells, which are especially adept at destroying cells infected with viruses. Another component of the innate immune system is the complement system. Complement proteins are normally inactive components of the blood. However, when activated by the recognition of a pathogen, the various proteins are activated to recruit inflammatory cells, coat pathogens to make them more easily phagocytosed, and to make destructive pores in the surfaces of pathogens. Other molecular components of innate responses include cytokines such as the interferons.

Methods described herein can be used to modulate innate immunity. Reducing the innate immunity response in a subject in need thereof, e.g., a subject exhibiting a pathogenically increased innate immunity response can be achieved by administering a TL1A blocking agent described herein. Increasing the innate immunity response in a subject in need thereof, e.g., a subject exhibiting an inadequate innate immunity response, can be achieved by administering a TL1A agonist agent, e.g., an anti-DR3 agonist antibody or other agonist described herein.

All references, including patent documents, disclosed herein are incorporated by reference in their entirety.

### EXAMPLES

### Example 1

### Role of TL1A in an Animal Model of Multiple Sclerosis

TL1A deficient mice were generated and were found to be phenotypically normal, with a unaltered distribution of immune cell subsets. We investigated the role of TL1A in MOG induced EAE, an animal model for multiple sclerosis (MS). We demonstrate that TL1A−/− animals have a lower incidence of EAE, a milder disease course and a lower level of inflammatory infiltrates in the CNS then wild type animals. TL1A deficient T cells have a comparable proliferative capacity but secrete lower levels of Th1 cytokines, especially IFN and GM-CSF in response to stimulation with MOG peptide. TL1A deficient T cells from MOG stimulated cultures also display a reduced level of cell surface markers and adhesion molecules characteristic of the effector T cell phenotype. These observations indicate that TL1A plays a role in the generation of MOG specific effector T cells and/or their ability to infiltrate and persist in the CNS and is a therapeutic target for treating MS.

### Generation of TL1A Deficient Mice

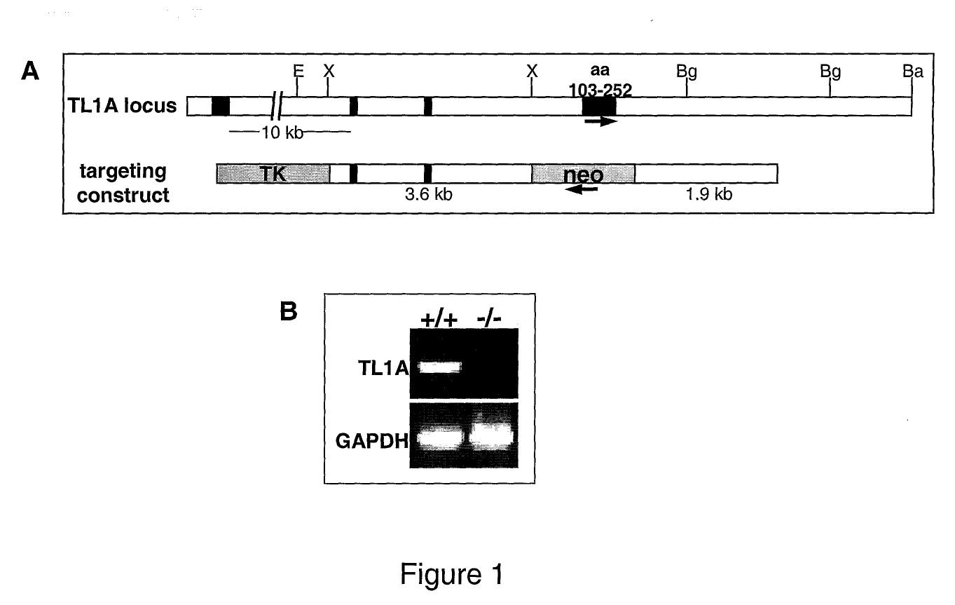

TL1A deficient mice were generated by replacing exon 4 of the TL1A (Tnfsf15) locus with a neomycin cassette (FIG. 1A). Exon 4 encodes amino acids 103-251 of TL1A encompassing the TNF-homology domain, essential for TL1A function. Lack of TL1A expression was confirmed by RT-PCR of kidney tissues (FIG. 1B) which contain high levels of TL1A mRNA. TL1A deficient mice were phenotypically normal and similar immune cell numbers and proportions were observed in the lymph nodes, spleen, thymus and bone marrow. Surface marker phenotype of lymph node cells is shown; no differences in marker expression were observed between TL1A^{-/-} and WT animals (not shown).

### Decreased Severity of EAE in TL1A^{-/-} Animals

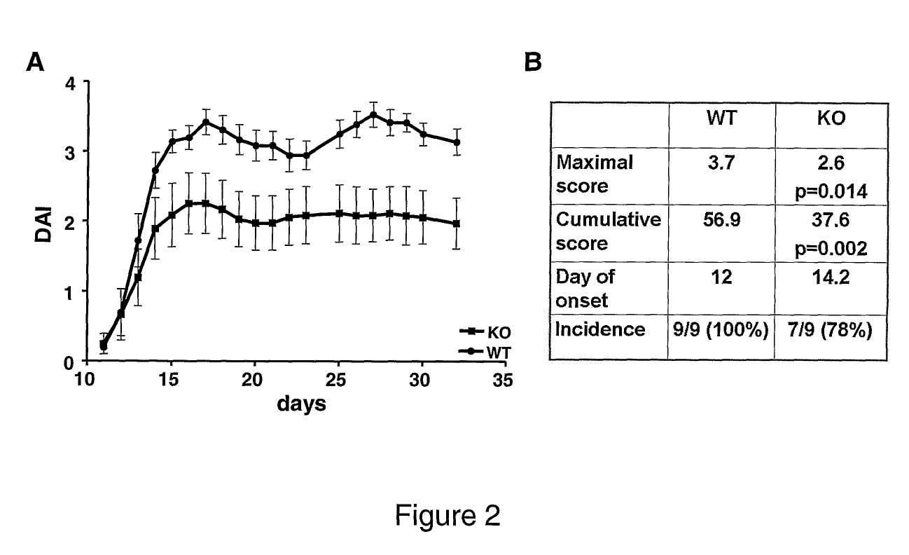

To determine the role of TL1A in the pathogenesis of MOG induced EAE, TL1A^{-/-} mice and WT controls were immunized s.c. with 200 μg of MOG₃₅₋₅₅ peptide and given 50 ng of pertussis toxin i.p. on the day of immunization. In four independent experiments TL1A^{-/-} and WT animals exhibited similar timing of disease onset. However TL1A deficient mice consistently showed a significantly reduced disease severity as manifested by a lower maximal disease score as well as lower scores throughout the course of the disease (FIG. 2). Disease incidence was similar in the two groups, with a consistent though not statistically significant, trend towards lower incidence in the TL1A^{-/-} mice.

TL1A^{-/-} Mice Show a Reduced Level of T Cell Infiltration into the CNS

Histological examination of the spinal cords was performed to determine whether the difference in clinical symptoms between TL1A^{-/-} and WT mice was reflected in the degree of inflammatory infiltration and demyelination in the CNS. TL1A^{-/-} mice exhibited fewer mononuclear infiltrates and demyelination foci than wild-type control animals at day 27 post-immunization. These results indicate that the observed reduced clinical disease in the KO animals is likely due to decreased CNS inflammation and damage. Since TL1A may be involved in the generation and/or function of MOG-specific T cells during the course of EAE, the levels of T-cell infiltration were quantified by image analysis of anti-CD3 staining. TL1A^{-/-} animals had fewer CD3 positive cells per spinal cord cross-section then wild type counterparts.

Inability to survive in the CNS is a possible mechanism underlying the reduction in T cell number in TL1A^{-/-} spinal cords. TUNEL and anti-activated caspase-3 staining were carried out to assess the extent of apoptosis in TL1A^{-/-} and wild type CNS. The level of apoptosis in the wild type spinal cord was low on days 21 or 27 post-immunization. Furthermore, no increase in apoptotic cells was observed in the KO spinal cords (not shown), suggesting that T cell apoptosis is unlikely to be a major mechanism behind the observed reduction in T cell infiltration of TL1A deficient CNS.

To examine whether the reduction in T cell frequency was manifest early in disease, levels of CD4⁺ T cells in the CNS (spinal cord and cerebellum) were assessed by flow cytometry. CD4⁺ T cells start accumulating in the CNS of WT mice one or two days prior to the onset of clinical symptoms and their levels peak at day 5-7 after disease onset. We found that the percentage of CD45⁺CD4⁺ cells in the TL1A^{-/-} CNS was consistently reduced as compared to WT CNS over the course of the study (not shown). Absolute numbers of CD45⁺CD4⁺ cell were also examined and showed a similar trend (not shown). These observations indicate that TL1A deficiency reduces and/or delays CD4 T cell infiltration into the CNS. The levels of CD45⁺CD11b⁺ cells in the CNS were comparable in TL1A deficient and WT animals (data not shown).

### TL1A is not Required for Antigen-Specific T-Cell Proliferation

TL1A expression on antigen presenting cells, such as macrophages has been suggested. Additionally, human recombinant soluble TL1A potentiates T cell responses under the conditions of suboptimal polyclonal stimulation in vitro. To examine whether the reduced clinical severity and T cell infiltration in TL1A^{-/-} mice is due to impaired antigen-specific T cell expansion, we used two independent systems. The role of TL1A in priming of naïve T cells was addressed using the OT-2 ovalbumin (OVA) specific TCR-transgenic system. CFSE-labeled naïve CD4⁺ T cells from OT-2 transgenic mice were transferred into TL1A^{-/-} or WT hosts. Twenty-four hours later 3 mg OVA protein and 5 ug LPS were administered by i.p. injection. Proliferation of OT-2 T cells in the spleens of recipient animals was examined 48 hrs subsequently. The pattern of CFSE dilution was independent of the genotype of the recipient animal, demonstrating that TL1A in not required for the priming of CD4⁺ T cells in this system.

To further examine whether antigen-specific T cells can proliferate in the TL1A knock-out (where both the T cells and the APCs lack TL1A) we studied the MOG-specific recall response. TL1A^{-/-} and WT animals were immunized with MOG₃₅₋₅₅ in CFA and in vitro T cell proliferation was examined on day 10. T cell proliferation in response to MOG₃₅₋₅₅ or anti-CD3 stimulation was comparable in lymph node cultures from TL1A^{-/-} and WT mice. The results from these two experimental systems indicate that TL1A does not play a significant role in CD4 T cell proliferation during initial priming or subsequent expansion of antigen-activated T cells.

TL1A Deficient T Cells have an Impaired Cytokine Response

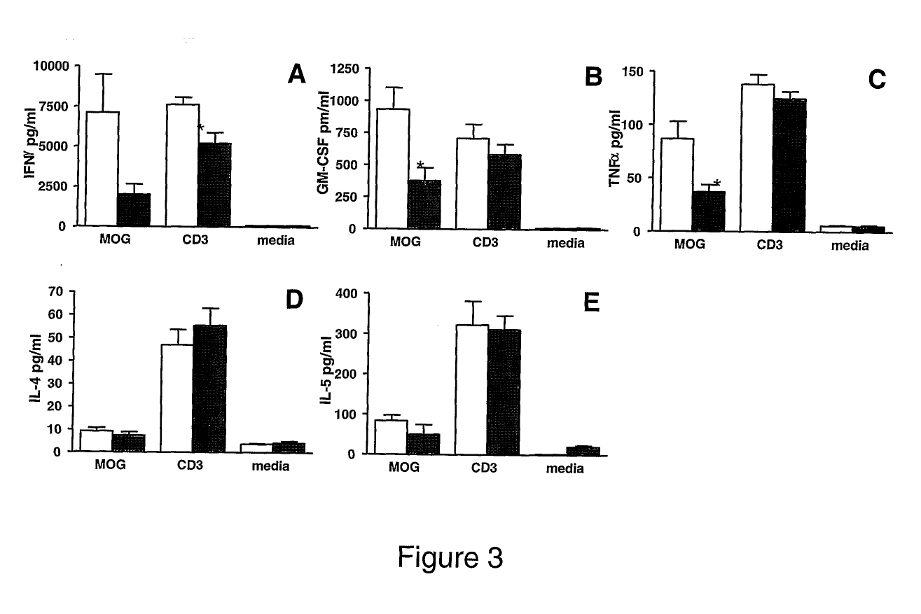

An alteration in the pattern on cytokines secreted by CD4⁺ T cells in TL1A^{-/-} mice may also lead to the observed amelioration of EAE. In the human system, treatment with soluble hTL1A has been reported to alter the pattern of cytokines secreted by activated T cells. To determine whether the absence of TL1A alters the T cell cytokine profile, MOG-specific responses were examined. TL1A^{-/-} and WT animals were immunized as above and levels of secreted cytokine from lymph node cultures were measured after 72 hrs. Consistent with the comparable proliferative response, the levels of T cell survival cytokine IL-2 were unaffected (data not shown). The levels of Th2-type cytokines IL-4 and IL-5 secreted in response to MOG₃₅₋₅₅ or anti-CD3 stimulation were comparable (FIGS. 3D, 3E). Levels of IL-10, IL-13 and IL-6 were similarly unaffected (data not shown). Interestingly, TL1A^{-/-} lymph node cultures secreted significantly lower levels of IFNγ, TNFα and GM-CSF in response to MOG stimulation as well as a lower level of IFNγ in response to anti-CD3 stimulation (FIGS. 3A, B and C). These observations suggest that TL1A deficiency impairs differentiation into Th1 cytokine producing effector cells, but does not appear to skew the response towards a Th2 phenotype.

T Cells from TL1A Deficient Animals Display at Altered Surface Marker Phenotype.

In addition to cytokine production, differentiation into effector T cells is reflected by a coordinated change in the pattern of surface molecules after antigen stimulation; the acquired pattern indicative of T cell activation and altered migratory capacity. Several of these molecules function in the homing of effector T cells out of the primary lymphoid organs and into the target tissue and may affect the ability to TL1A deficient T cells to infiltrate into the CNS. To examine the pattern of activation marker expression, TL1A^{-/-} and WT animals were immunized with MOG₃₅₋₅₅ peptide in CFA and draining LN cells cultured in the presence of MOG peptide, anti-CD3 or media alone and analyzed by FACS. Forward/side scatter profiles of the CD4⁺ cells), with gating on the larger CD4⁺ cells were analysed of the activated portion of the population. It should be noted that this population is not limited to MOG-specific activated T cells and likely contains bystander activated T cells as well. TL1A^{-/-} animals showed a small but significant decrease (mean value of 24.8±1.5% WT vs. 21.8±1.03% KO for cohorts of 5 animals) in the number of activated T cells on the basis of cell size recovered after culture.

Analysis of the activated CD4⁺ T cell population revealed an alteration in the surface marker profile in TL1A−/− as compared to WT cultures. Most notably, TL1A deficient cultures contained a larger percentage of cells expressing high levels of CD62L, the adhesion molecule present on naïve, lymph node resident T cells, which is downregulated with activation. TL1A−/− cultures exhibited a lower percentage of cells expressing E-selectin ligand, while the level of α4-integrin positive cells was somewhat increased in the KO. Expression of three other adhesion molecules CD44, LFA-1 and P-selectin ligand was unaffected. TL1A−/− cultures also showed a significant reduction as compared to WT in the percentage of cells expressing CD25 though not in their MFI values, as well as reduction in both the percent positive and MFI values for the early activation marker CD69. The expression of two co-stimulatory TNF family receptors was also examined. While the percentage of cells positive for OX40 was slightly but significantly lower in the absence of TL1A with an accompanying reduction in MFI, the pattern of CD27 expression was markedly altered, with higher levels observed on TL1A−/− cells, resembling a naïve phenotype. The overall alteration in surface marker profile indicates that antigen-activated TL1A−/− T cells do not acquire the full effector phenotype.

This study establishes a significant contribution of TL1A to the pathogenesis MOG-induced EAE and indicates that TL1A plays an important role in the acquisition of effector functions by T cells as evidenced by the altered pattern of secreted cytokines and surface markers.

### Example 2

### Role of TL1A in an Animal Model of UC and Innate Immunity

TL1A deficient mice were generated and were found to be phenotypically normal; with an unaltered distribution of immune cell subsets and apparently normal organ histology including colon. We investigated the role of TL1A in the DSS (dextran sodium sulfate) model of ulcerative colitis (UC) (see Dieleman et al. (1998) Clin. Exp. Immunol. 14:385-391). In this model, the colon is damaged by DSS inhibition of colonic epithelial proliferation, resulting in colonic ulcers, loss of the epithelial cell barrier and microbial activation of resident lamina propria immune cells and inflammation.

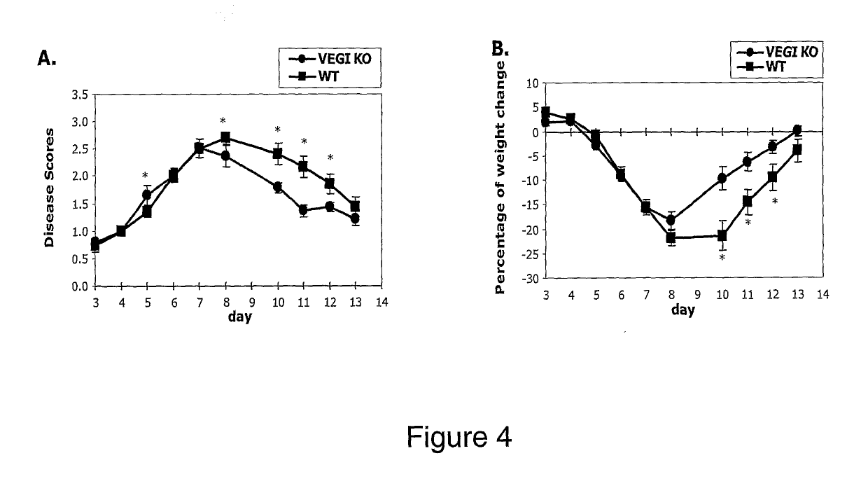

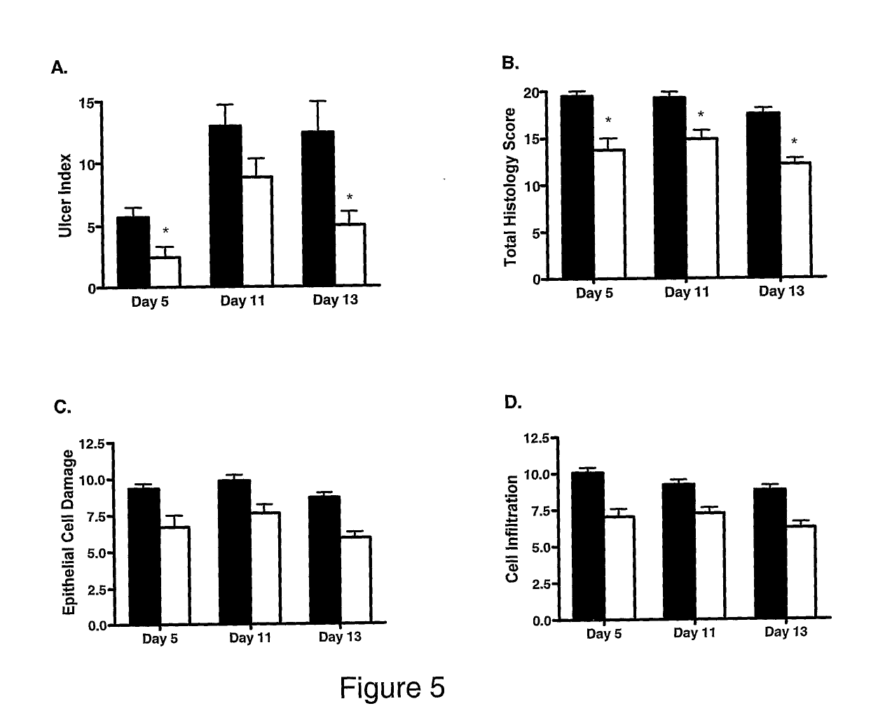

TL1A−/− animals were found to have a reduced severity of acute DSS colitis as compared to wildtype animals, as measured by reduced weight loss and clinical score (FIG. 4), as well as reduced histological score including ulcers, infiltration, goblet cell loss and crypt changes (FIG. 5). Immunohistochemical staining showed that the infiltrates associated with the ulcers included F4/80+ macrophages but not T lymphocytes (not shown). These data reveal a role for the TL1A pathway in the pathogenesis of UC and suggest that blocking TL1A can be useful to treat UC.

The ability to induce DSS colitis in RAG deficient mice which lack lymphocytes underscores the primary role of innate immune cell types and their release of proinflamrnatory cytokines, in the pathogenesis of this colitis. The data thus reveal a role for TL1A in promoting the innate inflammatory response. A Th1 or mixed Th1/2 response may occur in more chronic stages of the inflammation.

1. A method of treating multiple sclerosis in a subject, the method comprising administering to the subject a TL1A blocking agent selected from the group consisting of: (a) an anti-TL1A blocking antibody or antigen binding fragment thereof, (b) an anti-DR3 blocking antibody or antigen binding fragment thereof, (c) a soluble decoy DR3 polypeptide, (d) an anti-TL1A aptamer, (e) an anti-DR3 aptamer, (f) an RNAi inhibitor of TL1A, and (g) an RNAi inhibitor of DR3.

2. The method of claim 1, wherein the agent is an anti-TL1A blocking antibody or antigen binding fragment thereof.

3. The method of claim 1, wherein the agent is an anti-DR3 blocking antibody or antigen binding fragment thereof.

4. The method of claim 1, wherein the agent is a soluble decoy DR3 polypeptide.

5. The method of claim 1, wherein the agent is an anti-TL1A blocking antibody or an anti-DR3 blocking antibody, and wherein the antibody is a full length IgG.

6. The method of claim 1, wherein the agent is an antigen-binding fragment of an anti-TL1A blocking antibody or an anti-DR3 blocking antibody.

7. The method of claim 1, wherein the agent is an anti-TL1A blocking antibody or antigen binding fragment thereof, or an anti-DR3 blocking antibody or antigen binding fragment thereof, and wherein the blocking antibody or antigen binding fragment thereof is a single chain antibody, Fab fragment, F(ab′)₂ fragment, Fd fragment, Fv fragment, or dAb fragment.

8. The method of claim 1, wherein the agent is an anti-TL1A blocking antibody or antigen binding fragment thereof, or an anti-DR3 blocking antibody or antigen binding fragment thereof, and wherein the blocking antibody or antigen binding fragment thereof is a human, humanized or humaneered antibody.

9. The method of claim 4, wherein the polypeptide comprises a sequence which is at least 95% identical to amino acids 25-206 of SEQ ID NO:2 and binds TL1A.

10. The method of claim 4, wherein the polypeptide comprises a sequence which is at least 96% identical to amino acids 25-206 of SEQ ID NO:2 and binds TL1A.

11. The method of claim 4, wherein the polypeptide comprises a sequence which is at least 97% identical to amino acids 25-206 of SEQ ID NO:2 and binds TL1A.

12. The method of claim 4, wherein the polypeptide comprises a sequence which is at least 98% identical to amino acids 25-206 of SEQ ID NO:2 and binds TL1A.

13. The method of claim 4, wherein the polypeptide comprises amino acids 40-191 of SEQ ID NO:2 and binds TL1A.

14. The method of claim 4, wherein the polypeptide is fused to an Fc region of an Ig.

15. The method of claim 1, wherein the agent is administered in combination with a second therapeutic agent for multiple sclerosis.

16. The method of claim 15, wherein the second therapeutic agent is selected from the group consisting of: beta-interferon, copaxone, and natalizumab.

17. The method of claim 1, wherein the agent is an anti-TL1A blocking antibody or antigen binding fragment thereof, an anti-DR3 blocking antibody or antigen binding fragment thereof; or a soluble decoy DR3 polypeptide, and wherein the agent is administered at a dosage between 0.1-100 mg/kg.

18. The method of claim 1, wherein the agent is an anti-TL1A blocking antibody or antigen binding fragment thereof; an anti-DR3 blocking antibody or antigen binding fragment thereof; or a soluble decoy DR3 polypeptide and wherein the agent is administered via an intravenous, subcutaneous, intrathecal or intramuscular route.

19. A method of treating ulcerative colitis (UC) in a subject, the method comprising administering to the subject a TL1A blocking agent selected from the group consisting of: (a) an anti-TL1A blocking antibody or antigen binding fragment thereof, (b) an anti-DR3 blocking antibody or antigen binding fragment thereof, (c) a soluble decoy DR3 polypeptide, (d) an anti-TL1A aptamer, (e) an anti-DR3 aptamer, (f) an RNAi inhibitor of TL1A, and (g) an RNAi inhibitor of DR3.

20. The method of claim 19, wherein the agent is an anti-TL1A blocking antibody or antigen binding fragment thereof.

21. The method of claim 19, wherein the agent is an anti-DR3 blocking antibody or antigen binding fragment thereof.

22. The method of claim 19, wherein the agent is a soluble decoy DR3 polypeptide.

23. The method of claim 19, wherein the agent is an anti-TL1A blocking antibody or an anti-DR3 blocking antibody, and wherein the antibody is a full length IgG.

24. The method of claim 19, wherein the agent is an antigen-binding fragment of an anti-TL1A blocking antibody or an anti-DR3 blocking antibody.

25. The method of claim 19, wherein the agent is an anti-TL1A blocking antibody or antigen binding fragment thereof, or an anti-DR3 blocking antibody or antigen binding fragment thereof, and wherein the blocking antibody or antigen binding fragment thereof is a single chain antibody, Fab fragment, F(ab′)₂ fragment, Fd fragment, Fv fragment, or dAb fragment.

26. The method of claim 19, wherein the agent is an anti-TL1A blocking antibody or antigen binding fragment thereof, or an anti-DR3 blocking antibody or antigen binding fragment thereof, and wherein the blocking antibody or antigen binding fragment thereof is a human, humanized or humaneered antibody.

27. The method of claim 22, wherein the polypeptide comprises a sequence which is at least 95% identical to amino acids 25-206 of SEQ ID NO:2 and binds TL1A.

28. The method of claim 22, wherein the polypeptide comprises a sequence which is at least 96% identical to amino acids 25-206 of SEQ ID NO:2 and binds TL1A.

29. The method of claim 22, wherein the polypeptide comprises a sequence which is at least 97% identical to amino acids 25-206 of SEQ ID NO:2 and binds TL1A.

30. The method of claim 22, wherein the polypeptide comprises a sequence which is at least 98% identical to amino acids 25-206 of SEQ ID NO:2 and binds TL1A.

31. The method of claim 22, wherein the polypeptide comprises amino acids 40-191 of SEQ ID NO:2 and binds TL1A.

32. The method of claim 22, wherein the polypeptide is fused to an Fc region of an Ig.

33. The method of claim 19, wherein the agent is an anti-TL1A blocking antibody or antigen binding fragment thereof; an anti-DR3 blocking antibody or antigen binding fragment thereof; or a soluble decoy DR3 polypeptide, and wherein the agent is administered in combination with a second therapeutic agent for UC.

34. The method of claim 33, wherein the second therapeutic agent is selected from the group consisting of: corticosteroids, aminosalicylates, and immunosuppressants.

35. The method of claim 19, wherein the agent is an anti-TL1A blocking antibody or antigen binding fragment thereof; an anti-DR3 blocking antibody or antigen binding fragment thereof; or a soluble decoy DR3 polypeptide, and wherein the agent is administered at a dosage between 0.1-100 mg/kg.

36. The method of claim 19, wherein the agent is an anti-TL1A blocking antibody or antigen binding fragment thereof; an anti-DR3 blocking antibody or antigen binding fragment thereof; or a soluble decoy DR3 polypeptide and wherein the agent is administered via an intravenous, subcutaneous, intrathecal or intramuscular route.

37. A method of reducing an innate immunity response in a subject in need thereof, the method comprising administering, to the subject, an agent that blocks TL1A signaling, wherein the agent is selected from the group consisting of: (a) an anti-TL1A blocking antibody or antigen binding fragment thereof, (b) an anti-DR3 blocking antibody or antigen binding fragment thereof, (c) a soluble decoy DR3 polypeptide, (d) an anti-TL1A aptamer, (e) an anti-DR3 aptamer, (f) an RNAi inhibitor of TL1A, and (g) an RNAi inhibitor of DR3.

38. The method of claim 37, wherein the agent is an anti-TL1A blocking antibody or antigen binding fragment thereof.

39. The method of claim 37, wherein the agent is an anti-DR3 blocking antibody or antigen binding fragment thereof.

40. The method of claim 37, wherein the agent is a soluble decoy DR3 polypeptide.

41. The method of claim 37, wherein the agent is anti-TL1A blocking antibody or an anti-DR3 blocking antibody, and wherein the antibody is a full length IgG.

42. The method of claim 37, wherein the agent is an antigen-binding fragment of an anti-TL1A blocking antibody or an anti-DR3 blocking antibody.

43. The method of claim 37, wherein the agent is an anti-TL1A blocking antibody or antigen binding fragment thereof, or an anti-DR3 blocking antibody or antigen binding fragment thereof, and wherein the blocking antibody or antigen binding fragment thereof is a single chain antibody, Fab fragment, F(ab′)₂ fragment, Fd fragment, Fv fragment, or dAb fragment.

44. The method of claim 37, wherein the agent is an anti-TL1A blocking antibody or antigen binding fragment thereof or an anti-DR3 blocking antibody or antigen binding fragment thereof, and wherein the blocking antibody or antigen binding fragment thereof is a human, humanized or humaneered antibody.

45. The method of claim 40, wherein the polypeptide comprises a sequence which is at least 95% identical to amino acids 25-206 of SEQ ID NO:2 and binds TL1A.

46. The method of claim 40, wherein the polypeptide comprises a sequence which is at least 96% identical to amino acids 25-206 of SEQ ID NO:2 and binds TL1A.

47. The method of claim 40, wherein the polypeptide comprises a sequence which is at least 97% identical to amino acids 25-206 of SEQ ID NO:2 and binds TL1A.

48. The method of claim 40, wherein the polypeptide comprises a sequence which is at least 98% identical to amino acids 25-206 of SEQ ID NO:2 and binds TL1A.

49. The method of claim 40, wherein the polypeptide comprises amino acids 40-191 of SEQ ID NO:2 and binds TL1A.

50. The method of claim 40, wherein the polypeptide is fused to an Fc region of an Ig.

51. The method of claim 37, wherein the agent is an anti-TL1A blocking antibody or antigen binding fragment thereof, an anti-DR3 blocking antibody or antigen binding fragment thereof; or a soluble decoy DR3 polypeptide, and wherein the agent is administered at a dosage between 0.1-100 mg/kg.

52. The method of claim 37, wherein the agent is an anti-TL1A blocking antibody or antigen binding fragment thereof, an anti-DR3 blocking antibody or antigen binding fragment thereof: or a soluble decoy DR3 polypeptide, and wherein the agent is administered via an intravenous, subcutaneous, intrathecal or intramuscular route.

53. The method of claim 37, wherein the subject has an inflammatory disease or autoimmune disease.

54. The method of claim 37, further comprising evaluating the subject for a marker of innate immunity response.

55. A method of enhancing an innate immunity response in a subject in need thereof, the method comprising administering, to the subject, an agent that increases TL1A signaling.

56. The method of claim 55, wherein the agent is selected from the group consisting of: a soluble TL1A polypeptide, an anti-TL1A agonist antibody, and an anti-DR3 agonist antibody.

57. The method of claim 55, wherein the agent is a soluble TL1A polypeptide.

58. The method of claim 57, wherein the polypeptide comprises a sequence which is at least 95% identical to amino acids 103-251 of SEQ ID NO:1.

59. The method of claim 57, wherein the polypeptide comprises a sequence which is at least 96% identical to amino acids 103-251 of SEQ ID NO: 1.

60. The method of claim 57, wherein the polypeptide comprises a sequence which is at least 97% identical to amino acids 103-251 of SEQ ID NO:1.

61. The method of claim 57, wherein the polypeptide comprises a sequence which is at least 98% identical to amino acids 103-251 of SEQ ID NO: 1.

62. The method of claim 57, wherein the polypeptide is fused to a heterologous polypeptide.

63. The method of claim 62, wherein the heterologous polypeptide is an FC region of an Ig.

64. The method of claim 57, wherein the polypeptide is coupled to a non-polypeptide moiety.

65. The method of claim 64, wherein the non-polypeptide moiety is a chemical label or a lipid.

66. The method of claim 56, wherein the agent is administered at a dosage between 0.1-100 mg/kg.

67. The method of claim 55, wherein the agent is administered via an intravenous, subcutaneous, intrathecal or intramuscular route.

68. The method of claim 55, wherein the subject has a susceptibility to cancer.

69. The method of claim 55, wherein the subject has a family history of cancer.

70. The method of claim 55, wherein the subject has a genetic marker for cancer susceptibility.

71. The method of claim 55, wherein the subject has cancer.

72. The method of claim 55, wherein the subject has an opportunistic infection.

73. The method of claim 55, wherein the subject is exposed to radiation and/or one or more chemotherapeutic antiproliferative drugs.

74. The method of claim 55, wherein the subject has chronic respiratory disease or upper airways disease or chronic eye-ear-nose or throat infections.

75. The method of claim 55, wherein the subject is immunocompromised.

76. The method of claim 55, further comprising evaluating the subject for a marker of innate immunity response.


SEQUENCE LISTING (2 sequences)

SEQ ID NO: 1 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQGGDKLMVNVSDISLVDYTKEDKTFFGAFLL

SEQ ID NO: 2 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLTDVMDAVPAARWKEFVRTLGLREAEIEAVEVEIGRFRDQQYEMLKRWRQQQPAGLGAVYAALERMGLDGCVEDLRSRLQRGP